(19) United States
(12) Patent Application Publication
(10) Pub. No.: US 2024/0307338 A1
(43) Pub. Date: Sep. 19, 2024

(54) USE OF PHARMACEUTICAL COMPOSITION CONTAINING CHLOROGENIC ACID IN PREPARATION OF MEDICAMENT FOR TREATING PATHOLOGIC JAUNDICE

(71) Applicant: SICHUAN JIUZHANG BIOLOGICAL SCIENCE AND TECHNOLOGY CO., LTD., Sichuan (CN)

(72) Inventors: Jie ZHANG, Chengdu, Sichuan (CN); Xiaoguang CHEN, Chengdu, Sichuan (CN); Wang HUANG, Chengdu, Sichuan (CN); Yujia HUANG, Chengdu, Sichuan (CN)

(21) Appl. No.: 18/262,969

(22) PCT Filed: Jan. 13, 2021
(86) PCT No.: PCT/CN2022/071695

§ 371 Date: Jul. 26, 2023

(30) Foreign Application Priority Data
Jan. 27, 2021 (CN) 202110109946.1

(51) Int. Cl. A61K 31/216; A61K 31/702; A61K 35/00; A61K 35/745; A61P 1/16

(52) U.S. Cl. CPC A61K 31/216; A61K 31/702; A61K 35/745; A61P 1/16; A61K 2035/115

(57) ABSTRACT

A pharmaceutical composition containing chlorogenic acid is used in the preparation of a medicament for treating pathologic jaundice. In the pharmaceutical composition, chlorogenic acid is used as a main active ingredient, and bifidobacteria and human milk oligosaccharides are used as auxiliary ingredients. The latter can effectively enhance the therapeutic effect of chlorogenic acid for treating pathologic jaundice, thereby reducing the amount thereof. The pharmaceutical composition can effectively reduce the contents of total bilirubin and indirect bilirubin in blood, and provides a new means and choice for pharmacotherapy of pathologic jaundice, especially hemolytic jaundice.

### TECHNICAL FIELD

The present invention belongs to the field of biomedicine, and in particular, relates to the use of a pharmaceutical composition containing chlorogenic acid in the preparation of medicaments for treating pathological jaundice.

### BACKGROUND TECHNOLOGY

Jaundice is a common clinical symptom, especially in the neonatal period, and due to its different pathogenesis, it can be both a physiological phenomenon and a pathological phenomenon, in which hemolytic disease of the fetus and newborn (HDN), neonatal infection, biliary malformation, hepatitis and other diseases are the most common causes of pathological jaundice. According to the different causes of pathological jaundice, it usually includes hemolytic jaundice, hepatocellular jaundice, and obstructive jaundice. Among them, hemolytic jaundice is caused by the extensive destruction of red blood cells in a short period of time, and the release of bilirubin greatly exceeds the processing capacity of liver cells, resulting in jaundice. The increase in serum bilirubin is mainly caused by indirect bilirubin, and for example, neonatal jaundice and the jaundices caused by falciparum malaria or improper blood transfusion all belong to this category. Autoimmune hemolytic anemia, hereditary spherocytosis (HS), unstable hemoglobinopathy and other reasons lead to excessive destruction of red blood cells in the body, anemia, hemolysis, and excess raw materials for blood bilirubin, which can cause hemolytic jaundice.

Phenobarbital is a commonly used drug for the treatment of pathological jaundice, which can induce the activity of hepatic microsomal glucuronyl transferase, promote the combination of bilirubin and glucuronic acid, reduce the concentration of plasma bilirubin, and has obvious effect for the treatment of neonatal hyperbilirubinemia. However, after administration of phenobarbital, there are often residual effects such as dizziness and drowsiness, as well as side effects such as lethargy and slow milk sucking, and its long-term use can lead to resistance and dependence, and its repeated use is easy to produce cumulate toxicity; a few patients may experience allergic reactions such as rash, drug fever, and exfoliative dermatitis. In addition, blue light irradiation and Yinzhihuang can also be used to treat pathological jaundice in newborns. Blue light irradiation is a common method to treat neonatal jaundice. However, this method requires professional medical personnel to use professional equipment, which requires hospitalization for newborns, resulting in separation of mother and infant, and may affect breastfeeding. In addition, blue light irradiation treatment also has symptoms such as fever, rash, and diarrhea. Yinzhihuang is a traditional Chinese medicine for reducing jaundice, and consisted of Artemisiae Scopariae, Fructus Gardeniae, Scutellaria Baicalensis, Flos Lonicera, etc., which has the effects of clearing heat, detoxifying, removing dampness, and reducing jaundice. However, Yinzhihuang itself is a Chinese patent drug, and its ingredients are not clear, so it is difficult to evaluate its toxic and side effects. As early as 2016, the National Medical Products Administration banned the use of Yinzhihuang injections in newborns and infants. In 2017, a notice was issued to revise the instructions for Yinzhihuang oral formulations, which clearly emphasized the adverse reactions such as diarrhea, vomiting, and rash that may occur after taking Yinzhihuang orally. Therefore, developing new drugs for the treatment of pathological jaundice in infants, which are safe, effective, and easy to use, is of great significance.

Chlorogenic acid (CGA), also named caffetannic acid, is a phenolic acid composed of caffeic acid (CA) and quinic acid (QA), with a chemical name of 3-O-caffeoylquinic acid (CGA). Chlorogenic acid is a phenylpropanoid synthesized by the intermediate product of pentose phosphate pathway in the process of aerobic respiration of plants. CGA has been widely used in various fields, such as food, health care products, cosmetics and pharmaceuticals. CGA is widely present in various common vegetables and fruits, and has many biological activities, such as cardiovascular protection, antioxidant, anti-ultraviolet and anti-radiation, anti-mutation and anti-cancer, antibacterial and antiviral effects; lipid-lowering and hypoglycemic potentials; and immune regulatory activities, etc. CGA is widely used in medicine, chemical industry, food and other fields. It has been reported that CGA has a variety of pharmacological effects, and the present inventors have already reported for the first time that CGA has the effect of treating pathological jaundice.

The present invention is an improvement of the invention entitled “Use of chlorogenic acid in the preparation of medicament for treating pathological jaundice” (see CN104739818A, publication date: Jul. 1, 2015). In the in-depth study of the pharmacological effect of CGA, the present inventors have accidentally found a method that can significantly improve the efficacy of CGA in the treatment of pathological jaundice and reduce its amount, and thus have developed a pharmaceutical composition for the treatment of pathological jaundice, especially suitable for infants.

### CONTENT OF THE INVENTION

In order to solve the above technical problems, in the present invention, a safe and effective pharmaceutical composition for treating pathological jaundice, which is especially suitable for infants, is prepared by combining the main active ingredient CGA with the auxiliary ingredients bifidobacteria and human milk oligosaccharides in a specific dosage ratio.

In particular, the present invention has been performed by the following technical solutions:
in a first aspect, the present invention provides a pharmaceutical composition for the treatment of pathological jaundice, which comprises CGA, bifidobacteria and human milk oligosaccharides, and pharmaceutically acceptable excipients, and the weight ratio of CGA, bifidobacteria and human milk oligosaccharides is (5-10):(0.5-1):(0.5-1), wherein chlorogenic acid is the main active ingredient for the treatment of pathological jaundice, while bifidobacteria and human milk oligosaccharides are auxiliary ingredients used to enhance the efficacy of chlorogenic acid in treating pathological jaundice.

Alternatively, in the pharmaceutical composition mentioned above, the pathological jaundice is hemolytic jaundice, and preferably, the pathological jaundice is infant pathological jaundice, wherein the infant refers to children aged 0-12 months, preferably 0-6 months, and more preferably 0-3 months.

Alternatively, in the pharmaceutical composition mentioned above, the weight ratio of CGA, bifidobacteria and human milk oligosaccharides is 10:1:1.

Alternatively, in the pharmaceutical composition mentioned above, the human milk oligosaccharides are selected from one or more of 2′-fucosyllactose, 3′-fucosyllactose, lacto-difucotetraose, lacto-N-fucopentaose I, lacto-N-fucopentaose II, lacto-N-fucopentaose III, lacto-N-tetraose, lacto-N-neotetraose, 3′-sialyllactose, 6′-sialyllactose, sialyllacto-N-tetraose or disialyllacto-N-tetraose.

Alternatively, in the pharmaceutical composition mentioned above, the bifidobacteria are selected from one or more of Bifidobacterium longum, Bifidobacterium brevis, Bifidobacterium infantis, Bifidobacterium bifidum, Bifidobacterium adolescentis, Bifidobacterium pseudocatenulatum, and Bifidobacterium catenulatum.

Alternatively, in the pharmaceutical composition mentioned above, the pharmaceutical composition is an oral preparation, and the dosage form is selected from the group consisting of oral liquids, tablets, powder, capsules or granules.

Alternatively, in the pharmaceutical composition mentioned above, the pharmaceutical composition reduces the content of bilirubin in blood, promotes the biosynthesis of glutathione (GSH), and decreases the activity of α-glutathione-s-transferase (α-GST), alanine aminotransferase (ALT), and aspartate aminotransferase (AST).

In a second aspect, the present invention provides the use of the above pharmaceutical composition in the preparation of medicaments for the treatment of pathological jaundice.

Alternatively, in the use mentioned above, the pathological jaundice is hemolytic jaundice, and preferably, the pathological jaundice is infant pathological jaundice, in which the infant refers to children aged 0-12 months, preferably 0-6 months, and more preferably 0-3 months.

Alternatively, in the use mentioned above, the pharmaceutical composition reduces the content of bilirubin in blood, promotes the biosynthesis of glutathione (GSH), and decreases the activity of α-glutathione-s-transferase (α-GST), alanine aminotransferase (ALT), and aspartate aminotransferase (AST).

It should be understood that within the scope of the present invention, the above-mentioned technical features of the present invention and the specific technical features described below (such as in the example) can be combined with each other to form new or preferable technical solutions. Due to limited space, all of the technical solutions cannot be listed one by one herein.

Compared to the prior art, the present invention has the following beneficial effects:
(1) In the present invention, the main active ingredient CGA is combined with the auxiliary ingredients bifidobacteria and human milk oligosaccharides in a specific dosage ratio, and the latter can effectively enhance the therapeutic effect of CGA for treating pathologic jaundice, thereby greatly reducing the amount of CGA.
(2) The pharmaceutical composition of the present invention contains bifidobacteria and human milk oligosaccharides as auxiliary ingredients, that are commonly used in infant formula and nutritional supplements, and thus the pharmaceutical composition is very safe for infants, especially newborns.
(3) The pharmaceutical composition of the present invention can be orally administered, for example, it can be made into a preparation that can be added to infant formula or breast milk for taking, so as to facilitate home administration and increase infant compliance.

### EMBODIMENTS

In the in-depth study of the pharmacological effects of CGA, the present inventors have unexpectedly discovered a method that can significantly improve the efficacy of chlorogenic acid in the treatment of pathological jaundice and reduce its dosage by extensive screening, and developed a pharmaceutical composition particularly suitable for the treatment of pathological jaundice in infants.

On this basis, the present invention has been completed.

The term “infant”, as used herein, refers to children aged 0-12 months, preferably 0-6 months, and more preferably 0-3 months.

The term “human milk oligosaccharide (HMO)”, as used herein, denotes the third largest solid components in breast milk and has important physiological functions, including resistance to intestinal pathogens, regulation of immune response, and promotion of infant brain development. Due to the fact that most HMOs are not digested during gastrointestinal transit and thus can intactly reach the large intestine, they can promote the development and maturation of early gut microbiota in infants and young children, such as promoting the growth and colonization of bifidobacteria. The core structure of HMOs includes glucose (Glc), galactose (Gal), N-acetylglucosamine (GlcNAc), and further modified fucosyl (Fuc) and/or N-acetylneuraminic acid (Neu5Ac, sialic acid). According to the presence or absence of sialic acid, HMOs can be divided into two categories: neutral and acidic.

The term “human milk oligosaccharides (HMO)”, as used in the present invention, means one or more of 2′-fucosyllactose, 3′-fucosyllactose, lacto-difucotetraose, lacto-N-fucopentaose I, lacto-N-fucopentaose II, lacto-N-fucopentaose III, lacto-N-tetraose, lacto-N-neotetraose, 3′-sialyllactose, 6′-sialyllactose, sialyllacto-N-tetraose or disialyllacto-N-tetraose.

“Bifidobacteria”, as used herein, are a type of intestinal microbial ecological agents that, after oral administration, will grow and reproduce in the intestines of children, allowing beneficial microbiota to appear in the intestines of children in a short period of time and maintaining the normal microbiota in its optimal state.

The “bifidobacteria” used in the present invention are preferably human-derived bifidobacteria, which can be selected from one or more of Bifidobacterium longum, Bifidobacterium brevis, Bifidobacterium infantis, Bifidobacterium bifidum, Bifidobacterium adolescentis, Bifidobacterium pseudocatenulatum, and Bifidobacterium catenulatum.

The pharmaceutical composition of the present invention is an oral preparation, and the dosage form of the oral preparation is oral liquid, tablets, powder, capsules or granules. The preferred oral preparation is one that can be added to infant formula or breast milk.

The term “pharmaceutically acceptable excipients”, as used herein, refers to conventional drug carriers in the field of pharmaceutical preparations, and is selected from one or more of bulking agents, adhesives, disintegrants, lubricants, suspending agents, wetting agents, colorants, flavoring agents, solvents, and surfactants. Preferably, the “pharmaceutically acceptable excipients” of the present invention is a safe and non-toxic drug carriers suitable for infant use.

The bulking agents described in the present invention include but are not limited to starch, microcrystalline cellulose, sucrose, dextrin, lactose, icing sugar, glucose, etc.; the lubricants include but are not limited to magnesium stearate, stearic acid, sodium chloride, sodium oleate, sodium lauryl sulfate, poloxamer, etc.; the adhesives include but are not limited to water, ethanol, starch slurry, syrup, hydroxypropyl methyl cellulose, sodium carboxymethyl cellulose, sodium alginate, polyvinylpyrrolidone, etc.; the disintegrants include but are not limited to starch, effervescent mixture (namely sodium bicarbonate and citric acid), tartaric acid, low substituted hydroxypropyl cellulose, etc.; the suspending agents include but are not limited to polysaccharides such as acacia gum, agar, alginic acid, cellulose ether, and carboxymethyl chitosan; the solvents include but are not limited to water, balanced salt solutions, etc.

The various dosage forms mentioned above can be prepared according to conventional processes in the field of pharmaceutical preparations.

The present invention will now be described by reference to the following examples, which are merely illustrative and are not to be construed as a limitation of the scope of the present invention.

If the specific technology or conditions are not specified in the examples, they shall be carried out according to those described in the literature in this field or according to the product description. The reagents or instruments used without specifying the manufacturer are conventional products that can be commercially available.

The experimental methods in the following examples, unless otherwise specified, are all conventional methods. The experimental materials used in the following examples, unless otherwise specified, are all commercially available products.

Unless otherwise stated, the percentages and parts mentioned in the present invention are all weight percentages and weight parts.

### Example: In Vivo Pharmacodynamic Experiment on the Pharmaceutical Composition of the Present Invention Against Pathological Jaundice

### 1. Experimental Materials and Methods

### 1.1 Experimental Animals

Clean adult male SD rats, weighing 200 g-250 g.

### 1.2 Key Experimental Drugs and Reagents

Chlorogenic acid (the active pharmaceutical ingredient chlorogenic acid used in the example is extracted and purified from Eucommia ulmoides leaves, with a purity of more than 99.5%), Bifidobacterium brevis (M16-V), lacto-N-neotetraose, 6′-siallylactose, acetylphenylhydrazine (APH), total bilirubin test kit, direct bilirubin test kit, glutathione test kit, etc.

### 1.3 Establishment of Animal Models, Grouping, and Administration

The following experiment was carried out mainly by referring to the method described in CN104739818A. In short, in the example, acetylphenylhydrazine (APH) was used as a modeling drug to construct a rat model of hemolytic jaundice, which was also a commonly used model for studying neonatal hemolytic jaundice at home and abroad. The most prominent feature of hemolytic jaundice was the collapse of the patient's red blood cells, causing hemolysis. The drug used for modeling, acetylphenylhydrazine, had a slow progressive oxidative damage on red blood cells, making them prone to disintegration. At the same time, due to the rupture of red blood cells and the release of bilirubin, liver function was damaged. The mechanism and symptoms simulated by APH were similar to those of hemolytic jaundice.

From 60 healthy SD rats with required body weight, 10 rats were randomly selected as the normal control group, and the remaining 50 rats were intraperitoneally injected with acetylphenylhydrazine (APH) at a dose of 150 mg/kg for 3 consecutive days, to construct the animal model of hemolytic jaundice induced by oxidation of red blood cells. After that, blood was collected from the modeled rats and compared, and it was found that their bilirubin levels significantly increased compared to normal rats, indicating successful modeling, without rat death during the modeling process. 50 rats successfully modeled were randomly divided into 5 groups, 10 rats for each group, and the groups were respectively named as: model control group (n=10), CGA group (n=10), bifidobacteria+HMO group (n=10), CGA+bifidobacteria+HMO low-dose group (n=10), CGA+bifidobacteria+HMO high-dose group (n=10), together with the normal control group (n=10), a total of 6 experimental groups, including 60 rats.

In the experiment, rats in each group were administered by gavage, while the blank group and the model group were given the solvent physiological saline by gavage. The administration time is 6 days. The composition and dosage of therapeutic drugs for each group were as follows:
(1) CGA group: CGA 60 mg/kg/day, administered by gavage with physiological saline as solvent.
(2) bifidobacteria+HMO group: Bifidobacterium brevis M16-V (6 mg/kg/day), lacto-N-neotetraose (3 mg/kg/day), and 6′-sialyllactose (3 mg/kg/day). The mixture of the above components was prepared according to the weight ratio, and using physiological saline as the solvent, the solution of the mixture was administered by gavage.
(3) CGA+bifidobacteria+HMO low-dose group: CGA (60 mg/kg/day), Bifidobacterium brevis M16-V (6 mg/kg/day), lacto-N-neotetraose (3 mg/kg/day), and 6′-sialyllactose (3 mg/kg/day). The mixture of the above components was prepared according to the weight ratio, and using physiological saline as the solvent, the mixture solution was administered by gavage.
(4) CGA+bifidobacteria+HMO high-dose group: CGA (60 mg/kg/day), Bifidobacterium brevis M16-V (12 mg/kg/day), lacto-N-neotetraose (6 mg/kg/day), and 6′-sialyllactose (6 mg/kg/day). The mixture of the above components was prepared according to the weight ratio, and using physiological saline as the solvent, the mixture solution was administered by gavage.

### 2. Experimental and Testing Indicators

### 2.1 Detection of Liver Function Indicators

(1) After completion of the treatment experiment (for 6 consecutive days), blood was collected from each group of rats by removing the eyeballs and then labelled. Each blood sample was divided into three parts, in which one was used to test liver function indicators, and the other two were stored in a −20° C. refrigerator for future use. In this example, the activity of ALT (alanine aminotransferase) and AST (aspartate aminotransferase) was determined by rate method, while α-GST was detected using α-GST (α-glutathione s-transferase) detection kit;
(2) The blood sample stored in step (1) was taken out, and then bilirubin and direct bilirubin were detected by using the total bilirubin detection kit and the direct bilirubin detection kit.

### 2.2 Detection of Glutathione (GSH)

Glutathione plays an important role in maintaining the stability of erythrocyte membrane in the body. In order to explore the mechanism for the possible therapeutic effect of CGA, the GSH level of rats in each group was detected in this example. One part of the above frozen blood sample was subjected to the GSH detection using enzyme-linked immunosorbent assay (ELISA).

### 3. Statistical Processing

The experimental data with continuous variables were represented by mean±SD, and the comparison between the two sets of data was performed using two sample t-test. SPSS13.0 statistical software was used, and p<0.05 was considered statistically significant.

### 4. Experimental Results

### 4.1 the Detecting Results of Liver Function Indicators

The experimental results are shown in Tables 1 and 2 below.
(1) In the model group, the levels of α-GST, ALT, and AST in serum were significantly increased compared to the normal control group, and there was a significant difference between the two groups (p<0.05). The significant increase in the levels of α-GST, ALT, and AST is a pathological feature of hemolytic jaundice, indicating the successful establishment of pathological jaundice in rats.

In CGA group, the levels of α-GST, ALT, and AST in serum had significantly decreased after 6 days of treatment, and there was a significant difference compared with the model control group (p<0.05). In bifidobacteria+HMO group, after 6 days of treatment, the levels of α-GST, ALT, and AST in serum were only slightly lower than those in the model control group, indicating that treatment only with the combination of bifidobacteria+HMO did not improve pathological jaundice in experimental animals. In CGA+bifidobacteria+HMO low-dose group, the levels of α-GST, ALT, and AST in serum significantly decreased after 6 days of treatment, and the detection results of the above indicators were almost the same as those of the normal control group, and there was a significant difference compared to the model control group (p<0.01). The results showed that adding lower doses of bifidobacteria and HMO to CGA could significantly improve the therapeutic effect of CGA on pathological jaundice in experimental animals, compared with the results of CGA alone (p<0.05). In addition, in CGA+bifidobacteria+HMO high-dose group, the levels of α-GST, ALT, and AST in serum also significantly decreased after 6 days of treatment, and there was a significant difference compared to the model control group (p<0.05). However, interestingly, according to the results, although in the high-dose group, CGA was added with higher doses of bifidobacteria and HMO, the effect of CGA on pathological jaundice was not significantly enhanced, compared with the treatment group added low doses of bifidobacteria and HMO, and the testing results of the above indicators were almost the same as those of the CGA group.

It had been shown that the therapeutic effect of the pharmaceutical composition according to the present invention on pathological jaundice was not only related to the types of components, but also closely related to the weight ratio of components.

TABLE 1
Activity assay of α-GST, ALT, and AST in serum
of each group of rats (mean ± SD).

| Groups | α-GST | ALT(U/L) | AST(U/L) |
|---|---|---|---|
| Normal control group | 11.9 ± 3.1# | 18.7 ± 2.5# | 23.5 ± 3.7# |
| Model control group | 40.5 ± 2.6*Δ | 51.4 ± 3.9*Δ | 71.9 ± 4.8*Δ |
| CGA group | 20.9 ± 4.2# | 26.5 ± 3.8# | 29.8 ± 5.1# |
| Bifidobacteria + | 38.6 ± 2.9 | 50.8 ± 4.3 | 69.2 ± 5.6 |
| HMO group | | | |
| CGA + bifidobacteria + | 12.6 ± 1.9##Δ | 19.1 ± 2.3##Δ | 24.7 ± 3.1##Δ |
| HMO low-dose group | | | |
| CGA + bifidobacteria + | 20.4 ± 2.2# | 25.2 ± 3.1# | 28.9 ± 4.2# |
| HMO high-dose group | | | |

Note:
*p < 0.05, compared with the normal control group;
#p < 0.05, ##p < 0.01, compared with the model control group;
Δp < 0.05, compared with the CGA group.

(2) The detection results of direct bilirubin and indirect bilirubin showed that the level of total bilirubin (the sum of direct bilirubin and indirect bilirubin) in the model control group were significantly increased, with a significant difference compared to the normal control group (p<0.05). The release of bilirubin from the ruptured red blood cells caused the increase of the bilirubin level in the blood, which was a pathological feature of hemolytic jaundice, and these results indicated that the animal model of pathological jaundice in rats was successfully established.

In the CGA group, the levels of direct bilirubin, indirect bilirubin and total bilirubin had significantly decreased after 6 days of treatment, with a significant difference compared to the model control group (p<0.05). In the bifidobacteria+BMO group, the levels of direct bilirubin, indirect bilirubin, and total bilirubin were only slightly lower than those in the model control group after 6 days of treatment, indicating that treatment only with the combination of bifidobacteria+HMO did not alleviate pathological jaundice in experimental animals. In CGA+bifidobacteria+HMO low-dose group, the levels of direct bilirubin, indirect bilirubin, and total bilirubin significantly decreased after 6 days of treatment, and the detection results of the above indicators were almost the same as those of the normal control group, and there was a significant difference compared to the model control group (p<0.01). The results showed that adding lower doses of bifidobacteria and HMO to CGA could significantly improve the therapeutic effect of CGA on pathological jaundice in experimental animals, compared with the results of CGA alone (p<0.05). In addition, in CGA+bifidobacteria+HMO high-dose group, the levels of direct bilirubin, indirect bilirubin, and total bilirubin also significantly decreased after 6 days of treatment, and there was a significant difference compared to the model control group (p<0.05). However, interestingly, according to the results, although in the high-dose group, CGA was added with higher doses of bifidobacteria and HMO, the effect of CGA on pathological jaundice was not significantly enhanced, compared with the treatment group added low doses of bifidobacteria and HMO, and the results of the above indicators were almost the same as those of the CGA group.

Based on the above results, the therapeutic effect of the pharmaceutical composition according to the present invention on pathological jaundice was not only related to the types of components, but also closely related to the weight ratio of components.

TABLE 2
Determination of total bilirubin, direct bilirubin, and indirect
bilirubin in each group of rats (mean ± SD, μmol/L).

| | | | Indirect |
|---|---|---|---|
| Groups | Total bilirubin | Direct bilirubin | bilirubin |
| Normal control group | 1.79 ± 0.34# | 1.05 ± 0.37# | 0.74 ± |
| | | | 0.48# |
| Model control group | 7.80 ± 0.36*Δ | 3.78 ± 0.49*Δ | 4.02 ± |
| | | | 0.43*Δ |
| CGA group | 3.37 ± 0.93# | 1.86 ± 0.85# | 1.51 ± |
| | | | 0.78# |
| Bifidobacteria + | 7.66 ± 0.59 | 3.71 ± 0.41 | 3.95 ± |
| HMO group | | | 0.37 |
| CGA + | 1.85 ± 0.63##Δ | 1.08 ± 0.54##Δ | 0.77 ± |
| bifidobacteria + | | | 0.42##Δ |
| HMO low-dose group | | | |
| CGA + | 3.35 ± 0.82# | 1.87 ± 0.74# | 1.483.35 ± |
| bifidobacteria + | | | 0.71# |
| HMO high-dose group | | | |

Note:
*p < 0.05, compared with the normal control group;
#p < 0.05, ##p < 0.01, compared with the model control group;
Δp < 0.05, compared with the CGA group.

### 4.2 Detection Results of Glutathione (GSH)

In this example, the content of GSH in serum for each group of rats was detected. The results indicated that compared to the normal control group, the GSH levels in the model control group significant decreased, suggesting that insufficient GSH synthesis was a pathological feature of hemolytic jaundice. These results demonstrated that the animal model of pathological jaundice in rats was successfully established.

After treatment with CGA, the content of GSH in experimental animals increased significantly (p<0.05). However, in the bifidobacteria+BMO group, the GSH levels of experimental animals showed little change compared to the model control group. In CGA+bifidobacteria+HMO low-dose group, the GSH level increased significantly after 6 days of treatment, which was significantly different from the model control group (p<0.01). The results showed that adding lower doses of bifidobacteria and HMO to CGA could significantly improve the therapeutic effect of CGA on pathological jaundice in experimental animals, compared with the results of CGA alone (p<0.05). In CGA+bifidobacteria+HMO high-dose group, similar results were also obtained compared with CGA+bifidobacteria+HMO low-dose group, but its effect on the increase of GSH was not as good as the former (data not shown).

Based on the above experimental results, the pharmaceutical composition of the present invention containing CGA could effectively promote the synthesis of GSH, while GSH played a vital role in maintaining the stability of erythrocyte membrane. Therefore, the above results suggested that the pharmaceutical composition containing CGA might improve the hemolytic symptoms of hemolytic jaundice by promoting the synthesis of glutathione.

### 5. Experimental Conclusion and Discussion

In this example, a rat model of hemolytic jaundice had been successfully constructed, and various key indicators of the disease monitored in clinic were used as the testing indicators of this experiment, to investigate the in vivo effectiveness of the pharmaceutical composition according to the present invention containing CGA for the treatment of infant pathological jaundice.

The results indicated that in the present invention, the main active ingredient CGA was combined with the auxiliary ingredients bifidobacteria and HMO in a specific weight ratio, and bifidobacteria and HMO could effectively enhance the curative effect of CGA on pathological jaundice, thereby greatly reducing the amount of CGA.

The pharmaceutical composition of the present invention containing CGA could significantly improve the liver function of experimental animals, and effectively reduce the levels of α-GST, ALT, AST, as well as the content of total bilirubin, direct bilirubin, and indirect bilirubin in serum of model rats with pathological jaundice.

In addition, the pharmaceutical composition of the present invention containing CGA could significantly promote the synthesis of glutathione in rats of the experimental groups, suggesting that CGA might improve various abnormal indicators of hemolytic jaundice by promoting the biosynthesis of glutathione, thereby stabilizing the erythrocyte membrane and inhibiting the release of bilirubin.

Obviously, those skilled in the art could make various modifications and variations to the present invention, without departing from the spirit and scope of the present invention. In this way, if these modifications and variations of the present invention fall within the scope of the claims and their equivalents, the present invention is also intended to include these modifications and variations.

1. A pharmaceutical composition for the treatment of pathological jaundice, characterized in that the pharmaceutical composition comprises chlorogenic acid, bifidobacteria and human milk oligosaccharides, and pharmaceutically acceptable excipients, and the weight ratio of chlorogenic acid, bifidobacteria and human milk oligosaccharides is (5-10):(0.5-1):(0.5-1), wherein chlorogenic acid is the main active ingredient for the treatment of pathological jaundice, while bifidobacteria and human milk oligosaccharides are auxiliary ingredients used to enhance the efficacy of chlorogenic acid in treating pathological jaundice.

2. The pharmaceutical composition according to claim 1, characterized in that the pathological jaundice is hemolytic jaundice, and preferably, the pathological jaundice is infant pathological jaundice, wherein the infant refers to children aged 0-12 months, preferably 0-6 months, and more preferably 0-3 months.

3. The pharmaceutical composition according to claim 1, characterized in that the weight ratio of chlorogenic acid, bifidobacteria and human milk oligosaccharides is 10:1:1.

4. The pharmaceutical composition according to claim 1, characterized in that the human milk oligosaccharides are selected from one or more of 2′-fucosyllactose, 3′-fucosyllactose, lacto-difucotetraose, lacto-N-fucopentaose I, lacto-N-fucopentaose II, lacto-N-fucopentaose III, lacto-N-tetraose, lacto-N-neotetraose, 3′-sialyllactose, 6′-sialyllactose, sialyllacto-N-tetraose or disialyllacto-N-tetraose.

5. The pharmaceutical composition according to claim 1, characterized in that the bifidobacteria are selected from one or more of Bifidobacterium longum, Bifidobacterium brevis, infantis, Bifidobacterium bifidum, Bifidobacterium adolescentis, Bifidobacterium pseudocatenulatum, and Bifidobacterium catenulatum.

6. The pharmaceutical composition according to claim 1, characterized in that the pharmaceutical composition is an oral preparation, and the dosage form is selected from the group consisting of oral liquids, tablets, powder, capsules or granules.

7. The pharmaceutical composition according to claim 1, characterized in that the pharmaceutical composition reduces the content of bilirubin in blood, promotes the biosynthesis of glutathione (GSH), and decreases the activity of α-glutathione-s-transferase (α-GST), alanine aminotransferase (ALT), and aspartate aminotransferase (AST).

8. The use of the pharmaceutical composition according to claim 1 in the preparation of medicaments for the treatment of pathological jaundice.

9. The use according to claim 8, characterized in that the pathological jaundice is hemolytic jaundice, and preferably, the pathological jaundice is infant pathological jaundice, in which the infant refers to children aged 0-12 months, preferably 0-6 months, and more preferably 0-3 months.

10. The use according to claim 8, characterized in that the pharmaceutical composition reduces the content of bilirubin in blood, promotes the biosynthesis of glutathione (GSH), and decreases the activity of α-glutathione-s-transferase (α-GST), alanine aminotransferase (ALT), and aspartate aminotransferase (AST).
